(19) United States
(12) Patent Application Publication
(10) Pub. No.: US 2004/0158253 A1
(43) Pub. Date: Aug. 12, 2004

(54) Bracing device for fracture portions of bones

(76) Inventor: Shyang Cherng Liou, Chang Hua (TW)

Correspondence Address: Shyang Cherng Liou, P.O. Box 63-298, Taichung, 406, TW

(21) Appl. No.: 10/365,246

(22) Filed: Feb. 10, 2003

(51) Int. Cl. A61B 17/58

(52) U.S. Cl. 606/74

(57) ABSTRACT

A bracing device for bracing segments of bones together, includes a cylindrical barrel for receiving the segments of the bones therein, and one or more fasteners engaged through the cylindrical barrel for engaging into the segments of the bones, and to retain the segments of the bones together. The segments of the bones are normally formed beside the fracture portions of the bones, and may be solidly received and retained within the cylindrical barrel. The cylindrical barrel includes one or more orifices to receive the fasteners, and/or one or more openings to receive the newly grown bone tissue.

### BACKGROUND OF THE INVENTION

1. Field of the Invention

The present invention relates to a bracing device, and more particularly to a bracing device for bones of human beings or animals.

2. Description of the Prior Art

Typically, as shown in FIGS. 1 and 2, when bones 11 of limbs 10, such as legs, hands, or the like, of human beings or animals have fracture portions 111 formed therein, one or more nails 20 will be engaged through the separated parts or segments 12 of the bones 11, in order to secure and to retain the separated parts or segments 12 of the bones 11 together.

However, the separated parts or segments 12 of the bones 11 may not be solidly secured and retained together with these nails 20, such that the separated parts or segments 12 of the bones 11 may have a good chance to be separated or disengaged from each other again when forced are applied onto the limbs 10 or bones 11.

In addition, the nails 20 are normally engaged through the broken portions of the separated parts or segments 12 of the bones 11, such that the separated parts or segments 12 of the bones 11 may be easily further broken inadvertently.

The present invention has arisen to mitigate and/or obviate the afore-described disadvantages of the conventional bones securing devices or methods.

### SUMMARY OF THE INVENTION

The primary objective of the present invention is to provide a bracing device for solidly securing and retaining the separated parts or segments of the bones together.

In accordance with one aspect of the invention, there is provided a bracing device for bracing segments of bones together, the bracing device comprising a cylindrical barrel for receiving the segments of the bones therein, and at least one fastener engaged through the cylindrical barrel for engaging into the segments of the bones, and to retain the segments of the bones together. The segments of the bones are normally formed beside the fracture portions of the bones, and may be solidly received and retained within the cylindrical barrel.

The cylindrical barrel includes at least one orifice formed therein to receive the fastener, and may further include at least one opening formed therein, and having an inner diameter greater than that of the orifice of the cylindrical barrel for allowing the newly grown bone tissue to engage into the openings of the barrel, and for allowing the separated parts or segments of the bones to be further stably or solidly retained and positioned in the barrel.

Further objectives and advantages of the present invention will become apparent from a careful reading of the detailed description provided hereinbelow, with appropriate reference to the accompanying drawings.

### BRIEF DESCRIPTION OF THE DRAWINGS

FIGS. 1 and 2 are partial cross sectional views illustrating the securing of separated parts or segments of bones of limbs with typical nails;

FIG. 3 is a perspective view of a bracing device for bones in accordance with the present invention;

FIG. 4 is a partial cross sectional view of a bone or a limb having a bracing device in accordance with the present invention to secure or to retain the separated parts or segments of the bones together; and

FIGS. 5 and 6 are partial cross sectional views similar to FIG. 4, illustrating the securing or bracing of the separated parts or segments of the bones with the bracing device in accordance with the present invention.

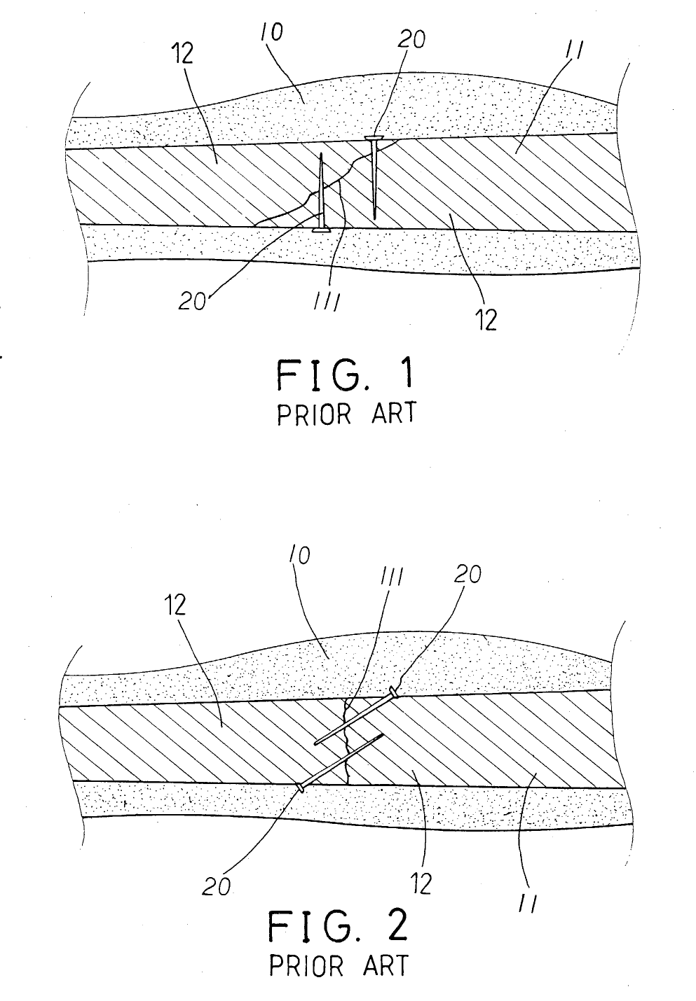

### DETAILED DESCRIPTION OF THE PREFERRED EMBODIMENT

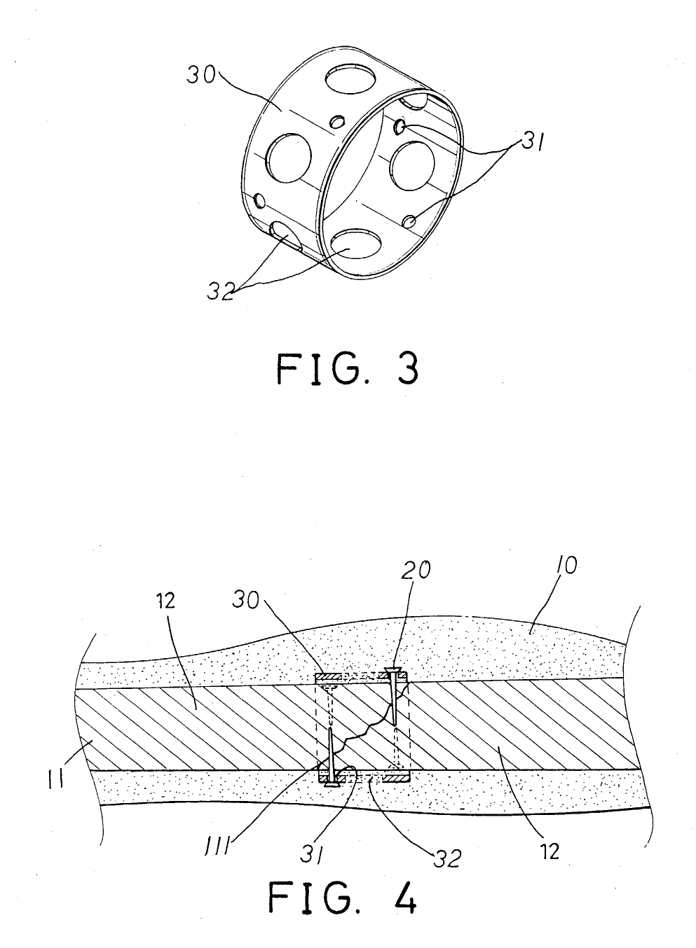

Referring to the drawings, and initially to FIGS. 3 and 4, a bracing device in accordance with the present invention comprises an annular or cylindrical barrel 30 for engaging onto or for receiving the separated parts or segments 12 of the bones 11 of the limbs 10 of such as human beings or animals. The separated parts or segments 12 of the bones 11 are normally formed beside one or more fracture portions 111 in the bones 11.

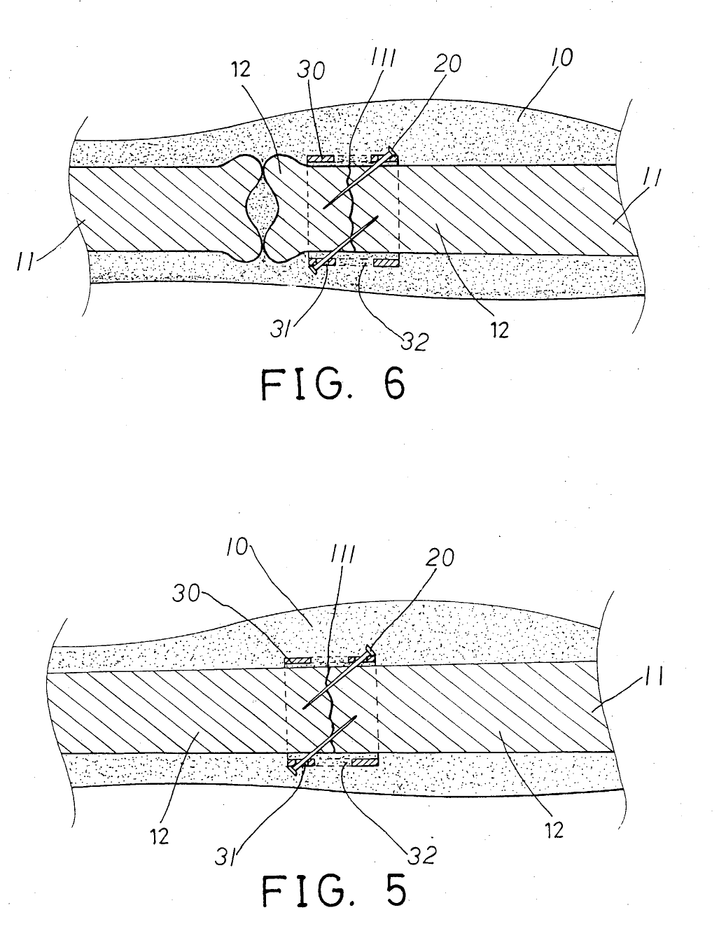

As shown in FIG. 4, the fracture portions 111 of the bones 11 may be inclined relative to the bones 11, or may be perpendicular to the bones as shown in FIGS. 5 and 6. In most of the typical fracture portions 111 of the bones 11, the separated parts or segments 12 of the bones 11 may be stably or solidly engaged in or retained in and positioned in the barrel 30, even when the bones 11 are crushed.

The cylindrical barrel 30 may be selected or formed to include an inner diameter slightly greater or smaller or close to the outer diameter of the bones 11, for allowing the separated parts or segments 12 of the bones 11 to be snugly retained in the barrel 30.

The barrel 30 includes one or more orifices 31 formed therein for receiving fasteners 20 which may engage through the separated parts or segments 12 of the bones 11, best shown in FIGS. 4-6, in order to further stably or solidly retain and position the separated parts or segments 12 of the bones 11 in the barrel 30.

It is preferable that the barrel 30 further includes one or more openings 32 formed therein, and preferably having inner diameters greater than that of the orifices 31 of the barrel 30, for allowing the newly grown bone tissue to engage into the openings 32 of the barrel 30, and for allowing the separated parts or segments 12 of the bones 11 to be further stably or solidly retained and positioned in the barrel 30.

In operation, as shown in FIG. 6, when the separated parts or segments 12 of the bones 11 include reduced lengths, the barrel 30 may also be used to stably or solidly retain and position the separated parts or segments 12 of the bones 11 in the barrel 30. The fasteners 20 may further solidly secure the separated parts or segments 12 of the bones 11 in the barrel 30.

Accordingly, the bracing device in accordance with the present invention may be used for solidly securing and retaining the separated parts or segments of the bones together.

Although this invention has been described with a certain degree of particularity, it is to be understood that the present disclosure has been made by way of example only and that numerous changes in the detailed construction and the combination and arrangement of parts may be resorted to without departing from the spirit and scope of the invention as hereinafter claimed.

I claim:

1. A bracing device for bracing segments of bones together, said bracing device comprising:
a cylindrical barrel for receiving the segments of the bones therein, and
at least one fastener engaged through said cylindrical barrel for engaging into the segments of the bones, and to retain the segments of the bones together.

2. The bracing device as claimed in claim 1, wherein said cylindrical barrel includes at least one orifice formed therein to receive said at least one fastener.

3. The bracing device as claimed in claim 2, wherein said cylindrical barrel includes at least one opening formed therein, and having an inner diameter greater than that of said at least one orifice of said cylindrical barrel.
